(19) United States
(12) Patent Application Publication
(10) Pub. No.: US 2006/0165782 A1
(43) Pub. Date: Jul. 27, 2006

(54) Solid compositions comprising gabapentin having improved stability

(76) Inventor: Bernard Charles Sherman, Toronto (CA)

Correspondence Address: NIXON & VANDERHYE, PC, 901 NORTH GLEBE ROAD, 11TH FLOOR, ARLINGTON, VA, 22203, US

(21) Appl. No.: 10/522,987

(22) PCT Filed: Aug. 6, 2003
(86) PCT No.: PCT/CA03/01174

§ 371 Date: Feb. 2, 2005

(30) Foreign Application Priority Data
Aug. 7, 2002 (CA) 2395931

(51) Int. Cl. A61K 33/42; A61K 31/195; A61K 9/20

(52) U.S. Cl. 424/464; 424/717; 514/561; 424/601

(57) ABSTRACT

Solid pharmaceutical compositions of improved stability which comprise gabapentin and a basic compound that is a hydroxide or a salt of a weak acid.

### BACKGROUND OF THE INVENTION

Gabapentin is a compound that is disclosed in U.S. Pat. Nos. 4,024,175 and 4,087,544, and is useful in therapy of certain cerebral disorders such as epilepsy.

Gabapentin is known to be susceptible to degradation into an impurity known as gabapentin lactam.

U.S. Pat. No. 6,054,482 discloses that in order to produce a stable composition comprising gabapentin it is necessary to do as follows:
1. Produce the gabapentin such that it contains less than 20 ppm of an ion of a mineral acid; and
2. Carefully select the excipients (inactive ingredients) used in the composition to exclude any excipient that catalyzes the degradation.

In the manufacture of hard gelatin capsules containing gabapentin, it is relatively simple to avoid use of excipients that catalyze degradation, because it is possible to fill capsules with gabapentin with no excipients at all, or with a minimal amount of excipients.

However, in the case of tablets comprising gabapentin, it is necessary to add a binder to give tablets of suitable hardness, as well as a lubricant to avoid sticking and binding in the tabletting process; and it is difficult if not impossible to find suitable excipients which enable satisfactory stability, especially if the gabapentin being used contains over 20 ppm of an ion of a mineral acid.

It is thus desirable to find a means of improving the stability of solid compositions that comprise gabapentin along with at least one excipient.

### DESCRIPTION OF THE INVENTION

It has been found that the stability of a solid composition comprising gabapentin can be significantly improved by inclusion of a relatively small amount of basic compound that is a hydroxide or a salt of a weak acid.

Compositions of the present invention thus are solid compositions comprising gabapentin, at least one excipient other than a basic compound that is a hydroxide or a salt of weak acid, and at least one excipient that is a basic compound that is a hydroxide or a salt of a weak acid, such as, for example, a carbonate, bicarbonate, or phosphate.

The basic compound will preferably be sodium hydroxide or a sodium salt of a weak acid, such as, for example, sodium carbonate, sodium bicarbonate, and tribasic sodium phosphate.

The amount of the basic compound relative to the amount of gabapentin by weight will preferably be under 5%, will more preferably be from about 0.01% to about 4%, and will even more preferably be from about 0.02% to about 1%.

The composition may be made by either a dry mix process (in which the ingredients are mixed without the use of a solvent) or by a wet granulation process in which a solvent is used and then evaporated.

The wet granulation process is preferable as it enables tablets of greater hardness.

The process will preferably include the steps of dissolving a binder (such as copolyvidone, povidone, or hydroxypropyl cellulose) in solvent, granulating the gabapentin with the solution, and drying to evaporate the solvent. The solvent may be water, but will preferably be or comprise an organic solvent, such as methanol, ethanol or methylene chloride.

The basic compound may be mixed with the gabapentin in dry form before the wet granulation is done. However, the basic compound will preferably be added to and mixed into the solution of the polymer in solvent before the solution is used to wet granulate the gabapentin.

After the granulation is complete and the solvent has been evaporated, the dried material will preferably be mixed with a lubricant, such as magnesium stearate, and optionally other excipients such as, for example, croscarmellose sodium as disintegrant.

The final mixture will then be compressed into tablets, which will optionally then be film-coated.

The invention will be better understood from the following examples, which are meant to be illustrative, and not limiting of the scope of the invention.

### EXAMPLE 1

Solutions A and B were prepared with ingredients in the following proportions:

Solution A:

| | |
|---|---|
| Copolyvidone | 24.9 parts |
| Methylene Chloride | 50.0 parts |
| | 74.9 parts |

Solution B:

| | |
|---|---|
| Sodium Carbonate anhydrous | 0.1 part |
| Water | 1.0 part |
| | 1.1 part |

Solution B was then added to and blended into Solution A and the resultant mixture was used to granulate 100 parts of gabapentin. After drying to evaporate the methylene chloride and water, the content of the dried mass was as follows:

| | | |
|---|---|---|
| Gabapentin | 100.0 | parts |
| Copolyvidone | 24.9 | parts |
| Sodium carbonate | 0.1 | part |
| | 125.0 | parts |

The stability of the resulting material was compared to that of material similarly prepared but without any sodium carbonate. This was done by measuring the increase in content of gabapentin lactam after storage at 60° C. for 48 hours.

The results as a percentage of the gabapentin were as follows:

| Sample | Increase in Lactam |
|---|---|
| Material of example 1 | 0.07% |
| Similar material without sodium carbonate | 0.16% |

It can thus be seen that the inclusion of the sodium carbonate significantly reduced the rate of increase of the lactam.

### EXAMPLE 2

Ingredients were mixed in the following proportions:

| | |
|---|---|
| Granules of example 1 | 1000 |
| Magnesium stearate | 3 |
| Croscarmellose sodium | 1 |
| | 1004 |

This mixture was compressed into tablets of weight 1004 mg each. Each tablet thus comprised 1000 mg of the granules of example 1, which in turn comprised 800 mg of gabapentin.

1. A solid pharmaceutical composition comprising gabapentin, a basic compound that is a hydroxide or a salt of a weak acid, and at least one other excipient that is not a hydroxide or a salt of a weak acid.

2. A composition of claim 1 wherein the basic compound is sodium hydroxide.

3. A composition of claim 1 wherein the basic compound is a sodium salt of a weak acid.

4. A composition of claim 1 wherein the basic compound is sodium carbonate.

5. A composition of claim 1 wherein the basic compound is sodium bicarbonate.

6. A composition of claim 1 wherein the basic compound is tribasic sodium phosphate.

7. A composition of claim 1 wherein the amount of the basic compound relative to the amount of gabapentin by weight is under 5%.

8. A composition of claim 1 wherein the amount of the basic compound relative to the amount of gabapentin is from about 0. 01% to about 4%.

9. A composition of claim 1 wherein the amount of the basic compound relative to the amount of gabapentin is from about 0.02% to about 1%.

10. A composition of claim 1 wherein made by a process in which a binder is dissolved in a solvent, the solution is used to wet granulate the gabapentin, and the solvent is evaporated.

11. A composition of claim 10 wherein the basic compound is added to the solution of the binder in the solvent.

12. A composition of claim 1, which comprises a binder, selected from copolyvidone, povidone and hydroxypropyl cellulose.

13. A composition of claim 1, which comprises copolyvidone as binder.

14. A composition of claim 1 in the form of a tablet.
